(19) United States
(12) Patent Application Publication
(10) Pub. No.: US 2006/0142356 A1
(43) Pub. Date: Jun. 29, 2006

(54) Novel therapeutic method and compositions for topical administration

(76) Inventors: Adrian Francis Davis, Weybridge, Surrey (GB); Lionel Trottet, Les Ulis (FR); Tadataka Yamada, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/545,772

(22) PCT Filed: Feb. 13, 2004
(86) PCT No.: PCT/US04/04393

§ 371 Date: Aug. 17, 2005

(30) Foreign Application Priority Data
Feb. 17, 2003 (GB) 0303600.1

(51) Int. Cl. A61K 31/426

(52) U.S. Cl. 514/369

(57) ABSTRACT

According to the invention there is provided a pharmaceutical composition suitable for topical administration to the skin which comprises farglitazar, or a pharmaceutically acceptable salt thereof, and a physiologically acceptable carrier and its use in the treatment of skin conditions.

This invention relates to a novel therapeutic method employing farglitazar, in particular to a method of treatment of diseases associated with the skin and to pharmaceutical compositions and their use in such method.

In the last decade or so a class of compounds known as thiazolidinediones (e.g. U.S. Pat. Nos. 5,089,514, 4,342,771, 4,367,234, 4,340,605, 5,306,726) have emerged as effective antidiabetic agents that enhance the insulin sensitivity of target tissues (skeletal muscle, liver, adipose) in animal models of non insulin dependent diabetes mellitus (“NIDDM”) and also reduce lipid and insulin levels in these animal models. The thiazolidinedione troglitazone was shown to have these same beneficial effects in human patients suffering from impaired glucose tolerance, a metabolic condition that precedes the development of NIDDM, as in patients suffering from NIDDM (J. J. Nolan et. al., N. Eng. J. Med. 1188-1193, 331 (1994)). While the mechanism of action is unclear, thiazolidinediones do not cause increases in insulin secretion or in the number or affinity of insulin receptor binding sites, suggesting that thiazolidinediones amplify post-receptor events in the insulin signaling cascade (J. R. Colca and D. R. Morton, “Antihyperglycemic thiazolidinediones: ciglitazone and its analogs,” in New Antidiabetic Drugs, C. J. Bailey and P. R. Flatt, eds., Smith-Gordon, New York, 255-261 (1990)).

Thiazolidinediones also induce the in vitro differentiation of preadipocyte cell lines into mature adipocytes (A. Hiragun, et. al. J. Cell. Physiol. 124-130, 134 (1988); R. F. Kleitzen, et. al., Mol. Pharmacol. 393-398, 41 (1992)). Treatment of pre-adipocyte cell lines with the thiazolidinedione pioglitazone results in increased expression of the adipocyte-specific genes aP2 and adipsin as well as the glucose transporter proteins GLUT-1 and GLUT-4, which suggests that the hypoglycaemic effects of thiazolidinediones seen in viva may be mediated through adipose tissue.

More recently, an orphan member of the steroid/thyroid/retinoid receptor superfamily of ligand-activated transcription factors termed Peroxisome Proliferator-Activated Receptor gamma (PPAR-gamma) has been discovered. PPAR-gamma is one of a subfamily of closely related PPARs encoded by independent genes (C. Dreyer, et. al., Cell 879-887, 68 (1992); A. Schmidt, et. al., Mol. Endocrinol. 1634-1641, 6, (1992); Y. Zhu, et. al., J. Biol. Chem. 26817-26820, 268 (1993); S. A. Kliewer et. al., Proc. Nat. Acad. Sci. USA 7355-7359, 91, (1994)). Three mammalian PPARs have been isolated and termed PPAR-alpha, PPAR-gamma, and NUC-1, or PPARδ. These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endocrin. Met. 291-296, 4 (1993)). Thiazolidinediones are now known to be potent and selective activators of PPAR-gamma and bind directly to the PPAR-gamma receptor (J. M. Lehmann et. al., J. Biol. Chem. 12953-12956, 270 (1995)), providing evidence that PPAR-gamma is a possible target for the therapeutic actions of the thiazolidinediones. Indeed, since PPAR-gamma was identified as a key molecular target for thiazolidinediones, this nuclear transcription factor has been identified in a large number of human cell types, and thiazolidinediones have been claimed to have a broad spectrum of potential clinical utilities, for example in certain forms of cancer (e.g. G. D. Demetri et al., Proc. Natl. Acad. Sci. USA 3951-3956, 96 (1999)), multiple sclerosis (e.g. M. Niino et al., Neuroimmunology 40-48, 116 (2001)), Alzheimers Disease (e.g. G. S. Watson and S. Craft, CNS Drugs 27-45, 17 (2003)), ulcerative colitis (e.g. J. D. Lewis et al, Am. J. Gastroenterology 3323-3328, 96 (2001)), asthma (Y. Hashimoto and K. Nakahara, Diabetes Care 401, 25 (2002)) and vascular disease (e.g. J. Minamikawa et al, J. Clin. Endocrinol. Metab. 1818-1820, 83 (1998)). Many potential disease targets for thiazolidinediones have an inflammatory component, and it is possible that it is the multi-faceted anti-inflammatory effects of these drugs which will prove to be of critical therapeutic importance. In this respect, it is now known that thiazolidinediones can modulate the functions of white blood blood cells (e.g. R. Garg et al, Hypertension 430-435, 36 (2000); N. Marx et al, Circ. Res. 703-710, 90 (2002)) as well as reduce their number in the circulation (S. M. Haffner et al, Circulation 679-684, 106 (2002)).

European Patent 306228 describes a class of PPAR gamma agonists which are thiazolidinedione derivatives for use as insulin sensitisers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound including the maleate salt are described in WO94/05659. European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione insulin sensitisers. Specific compounds that may be mentioned include 5-[4-[2-(5-ethyl-2-pyridyl)ethoxy]benzyl]thiazolidine-2,4-dione (also known as pioglitazone), 5-[4-[(1-methylcyclohexyl)methoxy]benzyl]thiazolidine-2,4-dione (also known as ciglitazone), 5-[[4-[(3,4-dihydro-6-hydroxy-2,5,7,8-tetramethyl-2H-1-benzopyran-2-yl)methoxy]phenyl]methyl]-2,4-thiazolidinedione (also known as troglitazone) and 5-[(2-benzyl-2,3-dihydrobenzopyran)-5-ylmethyl)thiazolidine-2,4-dione (also known as englitazone).

U.S. Pat. No. 6,294,580 (the disclosure of which is herein incorporated by reference in its entirety) describes a series of PPAR gamma agonist compounds not of the thiazolidinedione class but which are instead O— and N— substituted derivatives of tyrosine which nevertheless are effective as insulin sensitisers in the treatment of Type II diabetes mellitus. One such compound has chemical name N-(2-benzoylphenyl)-O-[2-(5-methyl-2-phenyl-4-oxazolyl)ethyl]-L-tyrosine (also known as 2(S)-(2-benzoyl-phenylamino)-3-{4-[2-5-methyl-2-phenyl-oxazol-4-yl)-ethoxy]-phenyl}-propionic acid, or by the generic name farglitazar, or by the registry number G1262570). A method of synthesising farglitazar is shown in Example 29.

U.S. Pat. No. 5,594,015 (Kurtz et al) describes the use of certain thiazolidinedione derivatives including pioglitazone and ciglitazone for the treatment of psoriasis through a mechanism involving inhibition of proliferation of keratinocytes. This patent describes a range of presentations by which the drug substance may be administered to the patient, including, for example, by applying a cream or oil of around 1-2% strength directly to the psoriatic lesion, or by administering the medication orally. U.S. Pat. No. 6,403,656 (Rivier et al) reports similar findings to those of Kurtz, together with the observation that the level of expression of PPAR gamma in psoriatic lesions is reduced relative to the healthy state. This patent describes the use of PPAR gamma agonists including thiazolidinediones as well as certain tyrosine derivatives in the treatment of abnormalities of differentiation in epidermal cells, more particularly in the treatment of psoriasis, atopic dermatitis and eczema, acne, light induced keratosis and skin cancers. The compounds are indicated for oral, topical and parenteral administration, for example by the topical route in the form of pasty ointments, creams, milks, creamy ointments, powders, impregnated pads, solutions, gels, sprays, lotions or suspensions typically at a concentration of 0.001-10% preferably 0.01-1% by weight based on weight of composition. Neither Kurtz nor Rivier discuss how to achieve an anti-psoriatic effect without at the same time causing an unwanted anti-hyperglycaemic effect.

Topical delivery of drugs provides a key advantage over systemic drug delivery as, ideally, the pharmacological effects of the drug administered will occur only locally and not systemically as plasma concentration will be too low to allow the drug to induce any pharmacological effect. This therefore offers the potential advantage of providing a larger therapeutic window with topical therapy than with systemic therapy.

Prior art in the topical drug development field does not however describe ways to quantify this therapeutic window. For oral treatment development, the quantification of the therapeutic window is classically achieved by using animal models and by identifying effective and toxic plasma levels in the animal. These data are then used to design a dose to be administered to human to generate effective but safe plasma levels. More recently, PharmacoKinetic/PharmacoDynamic models are starting to be used for drug development (“Opportunities for integration of pharmacokinetics, pharmacodynamics and toxicokinetics in rational drug development” p249-263 in ‘Integration of Pharmacokinetics, Pharmacodynamics, and Toxicokinetics in Rational Drug Development’—Editors: A. Yacobi, J. P. Skelly, V. P. Shah, L. Z Benet—1993—Plenum Press). However for topical drug development the use of such an approach has not been described. The likely reason for that is that such PK/PD models rely as a key factor on the determination of the concentration of drug in the tissue of interest. The direct determination of the local concentration of a drug applied topically is difficult for two main reasons. The first reason is due to local contamination of the sampled tissue as the surface and the superior layers of the skin contain an amount of drug several order of magnitude superior to the lower skin layers like the viable epidermis or dermis. The amount recovered in the sampled tissue is therefore likely to be overestimated by a factor unknown. The second reason concerns the determination within such a sample of the level of bound and unbound drug. This determination is indeed a requirement as only the unbound drug is considered to be able to cross biological membrane to induce its pharmacological action (“Pharmacokinetics and drug metabolism in animal studies” p23-31 of ‘Integration of Pharmacokinetics, Pharmacodynamics, and Toxicokinetics in Rational Drug Development’—Editors: A. Yacobi, J. P. Skelly, V. P. Shah, L. Z Benet—1993—Plenum Press). In plasma the measurement of the unbound fraction is relatively easily performed and the knowledge of this parameter is used to feed PK/PD models such as the Physiological Base Pharmacokinetic models. By contrast, in skin the determination of the unbound fraction is not easily determined.

As a result of these two issues, the determination of local skin concentration is not a factor that will really help the development of a topical compound. It should be noted that this measurement is none-the-less attempted because it is often considered as a regulatory requirement, but the scientist developing the topical treatment only knows that the experimentally determined concentration in the skin tissue should exceed the required effective concentration by a certain factor 2, 10, 100 or 1000 etc. He does not know the required value of this factor. Thus the ultimate test of knowing whether the treatment is effective or not and whether the drug is giving systemic side effect or not is really addressed only through the performing of actual clinical trials. Due to the absence of a manner of intelligently pre-selecting the desired dosage, and the need to be cautious in the performance of clinical trials which results in a tendency to under-dose, many drugs fail to achieve their therapeutic potential. Some drugs, such as PPAR gamma agonists, are capable of exhibiting a number of different therapeutic effects some of which will be desired and some of which will not be desired in a given clinical situation. For example one therapeutic effect may arise when the drug is administered topically and another when administered systemically. Thus there is needed a method of achieving the desired therapeutic effect without the undesired one.

According to the classical way of describing pharmaceutical compositions, whether they be oral or topical preparations, it is primarily the amount of drug present in these pharmaceutical compositions which is used to characterise the composition. For oral dosage forms, providing the drug is relatively well absorbed, the amount in the dose given is usually a good indicator of the dose absorbed and therefore of the plasma concentration. Variation in plasma levels achieved after the dosing of the same drug in two different oral preparations (absent the use of special sustained release technology) is usually small. In addition, for oral dosage forms, there is usually over a large dosing range a good proportional correlation between the dose given and the plasma level.

For topical dosage forms, the amount of drug present in the preparation is classically used to describe the preparation, as for oral preparations, and is usually expressed as the percentage of drug in the preparation. However, the variability in the amount absorbed is large compared to oral dosage forms as the bioavailability of topical drugs is generally low. This variability can be of one or two orders of magnitude depending on the excipients used in the preparation.

According to a first aspect of the invention we provide a pharmaceutical composition suitable for topical administration to the skin which comprises farglitazar, or a pharmaceutically acceptable salt thereof, and a physiologically acceptable carrier.

As a further aspect of the invention we provide use of such a formulation in the topical treatment of a skin condition such as psoriasis, atopic dermatitis, facial acne, rosacea, photoageing of the face, photoageing of the hands or skin cancer.

We also provide use of farglitazar, or a pharmaceutically acceptable salt thereof, in the manufacture of a medicament for the topical treatment of a skin condition such as psoriasis, atopic dermatitis, facial acne, rosacea, photoageing of the face, photoageing of the hands or skin cancer.

We also provide a method of treatment of a skin condition such as psoriasis, atopic dermatitis, facial acne, rosacea, photoageing of the face, photoageing of the hands or skin cancer which comprises administering topically to the skin such a formulation.

According to a particular aspect of the present invention we have also now invented a more reliable and predictable method of determining the therapeutic window for a topically administered pharmaceutical formulation which involves characterising that formulation in terms of the flux it delivers through skin. In particular the window is bounded at the upper end by a flux that is sufficiently low that it does not result in any undesired systemic pharmacological effect and is bounded at the lower end by a flux that is sufficiently high that it leads to a desired local pharmacological effect.

The prior art describes use of mathematical models to predict local concentration in epidermis or dermis (Kubota et al. (1993) J. Pharm. Sci. 82, 450-456; Nakayama et al. (1999) Pharm. Res. 16, 302-308; Parry et al. (1992) J.Invest. Derm. 98, 856-863; Mehta et al. (1997) J.Pharm. Sci 86, 797-801; Lee et al. (1993) Int. J. Pharm. 93,139-152; Imanidis et al. (1994) Pharm. Res. 11, 1035-1041; Roberts et al. “Mathematical models in percutaneous absorption” in ‘Percutaneous Absorption 3^{rd} edition’ Vol 97 of Drugs and the Pharmaceutical Science; Singh and Roberts (1993) J.Pharmacokinetics and Biopharm. 21, 337-373). However these documents do not use only flux as the key parameter for the whole assessment since other parameters need to be evaluated at the same time, such as partition coefficient, permeability coefficient or lag time. In Arzneim.Forsch./Drug Res. (2000) 50, 275-280, Wenkers and Lippold describe the use of flux and potency of NSAIDs to get to rank NSAIDs for topical efficacy but do not go further in defining how flux could be related to local concentration in the target site tissue or the likely expected clinical outcome. In European J. Pharmaceutics and Biopharmaceutics (2001) 51, 135-142, Cordero et al. describe a model that uses flux and potency of NSAIDs to get to a clinical effect as percentage of maximum pharmacological response. In this approach however, the target site is not clearly identified since NSAIDs are not classically use for dermis pain but are used for pain relief in deeper tissues. Also, the effect of the local clearance is not taken into account (i.e. only passive diffusion is addressed) and finally the effect of the disease on the local concentration is not considered. Imanidis et al (1994) Pharm Res 11(7) 1035-1041 reporting studies using topical acyclovir describes measurements of flux taken together with permeability coefficient to predict skin concentration, and reports a correlation between flux and efficacy in an animal model specific to herpes virus infection, but does not report any consideration of therapeutic index in drug treatment using acyclovir.

The prior art also describes use of flux through skin and of classical pharmacokinetics to predict plasma level following topical application (‘Investigations on the percutaneous absorption of the antidepressant rolipram in vitro and in vivo’—Hadgraft et al (1990) Pharm. Res. 7, 1307-1312), as it is the classical process used to develop a transdermal patch. Prior art as well describes the need when designing topical formulations to choose a compound with a high total systemic clearance (see eg “Discovery of ascomycin analogs with potent topical but weak systemic activity for treatment of inflammatory skin diseases” K. W. Mollison et al. (1998) Current Pharmaceutical Design 4, 367-379). The prior art does not describe the way to quantify the maximum flux to avoid systemic side effect by the four following factors: potency of the drug as the unbound concentration, the unbound fraction of the drug in plasma, the total systemic clearance of the drug and the classic body surface area treated for the skin disease concerned. It is therefore not described that for a specific skin disease both high total systemic clearance combined with small unbound fraction in plasma is required to increase the therapeutic window of a topical treatment.

In summary, none of the models referenced above has a similar approach to the one described in this invention.

The skin is well described in the literature [Monteiro-Riviere, N. A., 1991. Comparative anatomy, physiology, and biochemistry of mammalian skin. In: Hobson, D. W. (Ed.), Dermal and occular toxicology: Fundamentals and methods, CRC press, Boca Raton, pp. 3-71; Schaefer, H. and Redelmeier, T. E., 1996. Skin Barrier: Principles of percutaneous absorption, Karger, Basel]. In essence, as shown in FIG. 1, it comprises 3 main structures: the stratum corneum, the viable epidermis, the dermis, together with skin appendages; the follicles and sweat glands.

Stratum Corneum

The stratum corneum (SC) or horny layer, is the outermost layer of the skin and the main barrier to percutaneous absorption of chemical compounds despite being a very thin layer of an average thickness of 10-20 μm. The barrier properties of the SC are attributed to the highly organised layers of flattened, polygonal corneocytes and specialised intercellular lipids. The corneocytes are cell remnants of the terminally differentiated keratinocytes found in the basal layer of the epidermis at the epidermal-dermal junction. The corneocytes are surrounded by a practically insoluble and very resistant cell envelope. Around the corneocytes, the intercellular space is filled with lipids organised in stacked bilamellar structures sandwich with a continuous water phase. The lipids located in the intercellular space play a key role in the barrier formation.

The Viable Epidermis

Below the Stratum Corneum (SC), the main barrier to drug permeation sits the viable part of the epidermis. Its thickness varies from 50-200 μm. Its main function is the production and maintenance of the SC. It does have as well a role as a metabolic barrier against exogenous substances. The viable epidermis constitutes a dynamic system in which the keratinocytes, proliferated from the basal layer, differentiate as they progress towards the SC and get transformed into corneocytes. The turnover time for a keratinocyte from the basal layer to the skin surface is about 28 days for normal skin. It also contains specialised cells like melanocytes, which protect the body against UV radiation. The viable epidermis does not contain blood vessels as it receives nourishment from the dermis by passive diffusion. The viable epidermis is not considered as having strong barrier properties.

The Dermis

The dermis is situated below the viable epidermis. It is approximately 1 to 3 mm thick and makes the bulk of the skin. It consists of a matrix of connective tissue made from fibrous proteins like elastin and collagen. The main functions of the dermis are to give mechanical strength and elasticity to the skin barrier, to supply oxygen and nutrients, and to remove waste products. The dermis has an extensive vascular supply, which regulates temperature and pressure, delivers nutrients, removes waste products and mobilises defence forces. There are mainly two networks of blood vessels in the dermis: the superficial vascular plexus in the upper dermis and the deep vascular plexus in the lower dermis. These plexuses are extensively branched and a particularly dense network of capillaries is formed around the appendages. Due to the presence of these networks, exogenous substances as well as skin waste products are well cleared from the skin. Therefore the local concentration in the dermis of a compound applied topically is particularly low, and a steep concentration gradient from the skin surface to the dermis region is formed. The skin therefore acts very much as a “sink”.

The Skin Appendages

In humans, it is estimated that skin appendages account for less than 0.1% of the skin surface area but have to be considered in the discussion of percutaneous routes as they produce an apparent discontinuity in the barrier integrity. There are two types of skin appendages: the follicles and the sweat glands. Their structures have some similarities. They all cross the SC barrier and run deep in the dermis. They all have a central part believed to be relatively permeable. The outer part of these appendages is a membrane of unknown permeability. Unless this membrane is relatively permeable or can be opened under certain condition, the skin appendages will or will not constitute a route of entry of exogenous compounds.

The Follicles

There are three types of follicles in human skin:
Terminal hair follicles (e.g. hair on the head) in which a large hair is associated with a large sebaceous gland. The root of this hair may extend more than 3 mm below the skin surface into the subcutaneous fatty tissue.
Vellus hair follicles (e.g. fine hairs on the face in women), in which only a small sebaceous gland is associated with a fine hair. Its roots extend less than 1 mm in the dermis.
Sebaceous follicles (or sebaceous gland follicles), characteristics of human beings and not present in animals. They are found mainly on the face and the central parts of chest and back. It consists of 4 parts: the sebaceous gland lobes that secretes sebum (a mixture of lipids), the sebaceous ducts that connects the lobes and the secretory duct, a small vellus hair and the secretory duct which is a long duct lined by keratinocytes that is the large conducting duct that conducts the sebum coming from the sebaceous gland to the skin surface. The keratinocytes produce corneocytes which normally are ejected outwards. The sebaceous duct is situated approximately 0.5 mm below the skin surface.
   The Sweat Glands

Sweat glands are tubular glands distributed almost over the entire human body. Each gland has a secretory part located below the dermis in the subcutaneous tissue and an excretory duct that ultimately opens directly on the skin surface. These glands produce perspiration.

A number of skin conditions may desirably be treated by topical administration of an active drug substance. For example:

Psoriasis is an inflammatory skin condition which has been described in J Invest Dermatol (1983) 81 503-506 and J Amer Acad Dermatol (1983) 8(5) 645-647. The psoriatic skin lesions are inflammatory, red, sharply delimited plaques of various shapes with characteristics silvery lustrous scaling. There are two primary activities affecting the psoriasis skin condition. The first one occurs in the epidermis. There is a large increase in the volume of the epidermis which is 4 to 6 fold greater than normal characterised by high keratinocyte proliferation and a very short keratinocyte life cycle—from basal layer to stratum corneum shedding—which is reduced to about 2 days as opposed to 28 days for normal skin. The second one occurs in the dermis where there is a strong inflammatory reaction coupled with a network of capillaries expanding from the one present in normal skin (this is called angiogenesis) [Braun-Falco O., Plewig G., Wolff H. H. & Winkelmann R. K. (1991) Erythematous and erythematosquamous skin diseases In: Dermatology (Braun-Falco O., Plewig G., Wolff H. H. & Winkelmann R. K., eds.), Springer-Verlag press, Berlin, Heidelberg, New York]. The target layer of the skin is usually both the bottom of the epidermis and the top of the dermis. For example in the case of treatment by PPAR gamma agonists, both the bottom of the epidermis and the top of the dermis are considered the target sites for the drug's effect, as such drugs can affect (a) the differentiation/proliferation of keratinocytes process (bottom of epidermis), (b) the inflammatory process (top of dermis), and (c) the angiogenesis process (top of dermis).

Atopic dermatitis is an inflammatory skin condition which has been described in J Invest Dermatol (1982) 79 243-245, Br J Dermatol (1988) 118, 517-522, J Am Acad Dermatol (1995) 33, 969-972 and J Dermatol Sci (2000) 23,178-182—“Atopic Dermatitis: From Pathogenesis to treatment”—author/editor Leung D. Y. M.—1996—-Springer-Verlag. Atopic dermatitis is the most common form of dermatitis and is a chronic inflammatory skin condition whose classic feature is itchy skin. The great majority of cases occur in infancy and childhood. The skin is dry, flaky, rough, and can be secondarily infected and show oozing and crusts. The skin barrier property of atopic dermatitis lesions is viewed as being down compared to normal. The main treatment skin tissue target for this disease is the top layer of the dermis where the inflammation occurs. For example, in the case of treatment by PPAR gamma agonists, the top of the dermis is considered the target for the drug's effect, as such drugs can affect the inflammatory process (top of dermis).

Facial acne is one of the most common diseases in dermatology. It occurs at puberty in almost every one, although to different extents and regresses in early adulthood. The preferred sites are naturally on the face as this is a disease affecting the sebaceous follicles. It is characterised by seborrhoea, disturbed keratinisation in the follicles with comedones and subsequent inflammatory papules, pustules, and nodular abscesses and scars. One of the most important factors in acne is the superior production of sebum in acne sites as opposed to healthy skin sites. Sebaceous follicles are densely populated by bacteria and fungi. In the deeper anaerobic area of the sebaceous glands one finds propionibacteria. These produce lipases and are regarded as a substantial factors in the pathogenesis of acne. The first detectable sign of acne is increased production of corneocytes in the secretory duct, but these are no longer extruded outwards. Comedones (blockage of the sebaceous follicle) occur therefore through hyperkeratosis associated with proliferation and retention. Bacteria proliferation and inflammation can then occur [Braun-Falco O., Plewig G., Wolff H. H. & Winkelmann R. K. (1991) Disease of the sebaceous follicles In: Dermatology, Springer-Verlag press, Berlin, Heidelberg, New York.] For example in the case of treatment by PPAR gamma agonists, the whole dermis is considered the target for the drug's effect, as such drugs can affect (a) the differentiation/proliferation of keratinocyte that cover the wall of the sebaceous ducts, which ducts run down into the dermis (whole dermis), (b) the inflammatory process that occurs around the sebaceous gland (whole dermis), and (c) the excess sebum production (on the external surface of sebocyte gland) (whole dermis).

Rosacea is a condition having as a classic symptoms a persistent flushing of the face, leading to a polymorphic picture of persistent redness, papules, telangiectasia, thickening and coarsening of the skin and in its most extreme form gross enlargement and deformity of the nose. The areas most characteristically affected are the central convex areas of the face (nose, forehead, cheeks and chin), although the scalp, upper chest, back and even the limbs may be involved. The onset of rosacea is preceded by episodic flushing unaccompanied by sweating, these attacks being readily triggered by local stimuli including hot drinks, spicy food, alcohol, excessive sunlight and emotion. Erythema, which is accompanied by a burning sensation, gradually becomes more persistent and is associated with increasingly prominent telangiectasia. More advanced stages show follicular and non-follicular papules and pustules without comedones followed by persistent tissue thickening due to oedema leading ultimately to a peau dorange appearance and rhinophyma. Untreated, rosacea becomes slowly but inexorably more severe, although there may be temporary remissions and severe relapses on the way depending on the exposure to or withdrawal of exacerbating factors. For example in the case of treament by PPAR gamma agonists, the top of the dermis is considered as a target site for the drug's effect, as such drugs can affect (a) the inflammatory process (top of dermis), and (b) the angiogenesis process (top of dermis).

Skin Photoageing is the gradual deterioration of cutaneous structure and function following long term, recurrent exposure to sunlight or artificial UVR sources. It apparently occurs as a result of cumulative DNA damage resulting from recurrent, acute DNA injury and from the effects of chronic inflammation. Epidermis and dermis are both affected by UVB and the dermis is significantly affected by UVA as well. Histologically, there is a mild inflammatory infiltrate and profuse upper dermal accumulation of a form of amorphous, degenerate elastic tissue, known as elastosis. Both are probably responses to UVR-induced elastin promoter, metalloproteinase and cytokine activation. In addition, and probably for the same reason, dermal collagen is also degenerated and somewhat diminished in amount. [“Disorders of connective tissue” by Burton J. L., Lovell C. R. Chapter 44 as well “Cutaneous Photobiology” by Hawk J. L. M. Chapter 25 in “Textbook of Dermatology” by Rook A., Wilkinson D. S., Ebling F. J. G., edited by Champion R. H., Burton J. L., Burns D. A., Breathnach S. M.—6^{th} Edition—1998—Publisher: Blackwell Science; “A review of skin ageing and its medical therapy”, Gilchrest B. A., British Journal of Dermatology, 1996, 135, 867-875]. Overall, the dermis is the main skin target site for drug therapy. For example in the case of treament by PPAR gamma agonists, the whole dermis is considered as the target site for the drug's effect, as such drugs can affect (a) the differentiation/proliferation of the poorly differentiated cells of the dermis (whole dermis), (b) the inflammatory process (top of dermis), (c) the production of collagen break down product (whole dermis) and (d) the Retinoid X Receptors (whole dermis).

Skin cancer can be present in many forms that can affect all the viable skin tissues (epidermis, dermis or appendages). The different forms can be benigm or malignant tumours. Their diversity is described in the 3 chapters of Textbook in Dermatology [“Epidermal skin tumours” by McKie R. M. Chapter 36 as well “Tumours of the skin appendages” by McKie R. M. Chapter 37 as well “Soft-Tissue Tumours” by McKie R. M. Chapter 55 in “Textbook of Dermatology” by Rook A., Wilkinson D. S., Ebling F. J. G., edited by Champion R. H., Burton J. L., Burns D. A., Breathnach S. M.—6^{th} Edition—1998—Publisher: Blackwell Science]. As in all tumours, their main characteristics is a proliferation of cells which is coupled by a substantial increase of local vasculature. The target site is therefore the whole depth of the skin viable tissue, ranging from the epidermis to the bottom of the dermis. For example in the case of treament by PPAR gamma agonists, the bottom of the epidermis as well as the whole dermis are considered as the target site for the drug's effect, as such drugs can affect (a) the differentiation/proliferation of skin cells [melanocytes in particular] (bottom of epidermis and whole dermis) and (b) the angiogenesis process (top of dermis).

Thus according to the invention there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment a pharmaceutical composition containing a drug substance which is farglitazar or a pharmaceutically acceptable salt thereof characterised in that the formulation delivers to the skin a nominal flux of said drug substance which is greater than the minimum nominal flux required to cause a therapeutic effect against the skin condition (hereinafter “lower limit nominal flux”) and less than the minimum nominal flux which will cause a systemic anti-hyperglycaemic effect (hereinafter “upper limit nominal flux”).

The minimum nominal flux which will cause a systemic anti-hyperglycaemic effect (“upper limit nominal flux”) is defined in this invention as the flux which results in a peak plasma concentration which is comparable with (for example the same as or within around 20%, ideally the same as) that obtained by administering farglitazar with a therapeutic dose by the oral route (“oral therapeutic dose”). An oral therapeutic dose is defined as a dose that would lead to a substantial systemic anti-hyperglycaemic effect if dosed to a patient suffering from Type II diabetes mellitus or another condition alleviated by insulin sensitivity enhancement. For the avoidance of doubt, however, the method is not limited to the treatment of patients suffering from such conditions.

According to another aspect of the invention there is provided use of a pharmaceutical composition containing a drug substance which is farglitazar or a pharmaceutically acceptable salt thereof in the topical treatment to the skin of a patient suffering from a skin condition selected from the list consisting of psoriasis, atopic dermatitis, facial acne, rosacea, photoageing of the face, photoageing of the hands, and skin cancer characterised in that the composition delivers to the skin a nominal flux of said drug substance which is greater than the minimum nominal flux required to cause a therapeutic effect against the skin condition and less than the minimum nominal flux which will cause a systemic anti-hyperglycaemic effect.

There is also provided use of a pharmaceutical composition containing a drug substance which is farglitazar or a pharmaceutically acceptable salt thereof in the manufacture of a medicament for the topical treatment to the skin of a patient suffering from a skin condition selected from the list consisting of psoriasis, atopic dermatitis, facial acne, rosacea, photoageing of the face, photoageing of the hands, and skin cancer characterised in that the composition delivers to the skin a nominal flux of said drug substance which is greater than the minimum nominal flux required to cause a therapeutic effect against the skin condition and less than the minimum nominal flux which will cause a systemic anti-hyperglycaemic effect.

As discussed in the introduction above, the relationship between the flux of drug substance delivered by a topical formulation and the concentration of drug substance within that formulation is complicated by the effect of the other formulation ingredients. As a result the therapeutic effect and risk of adverse event, or unwanted pharmacological effect, is not readily judged based on purely the concentration of drug substance in a given topical formulation. We have however, as an aspect of the invention, also invented a method in which a suitable and safe flux of drug substance may be determined for use in treating a range of skin conditions based on knowledge of only a few fundamental drug parameters.

Thus according to this aspect of the invention, the lower limit nominal flux and the upper limit nominal flux are respectively given (expressed in units of ng/cm²/hr) by equations (1) and (2) as follows:
(a) in the case of psoriasis equation (1) is:
   Z/2.4
and equation (2) is:
   Z*C/[200*(100-PBF)]
(b) in the case of atopic dermatitis psoriasis equation (1) is:
   Z/6
and equation (2) is:
   Z*C*/[600*(100-PBF)]
(c) in the case of facial acne equation (1) is:
   Z/10
and equation (2) is:
   Z*C*/[200*(100-PBF)]
(d) in the case of rosacea equation (1) is:
   Z/10
and equation (2) is:
   Z*C*/[200*(100-PBF)]
(e) in the case of photoageing of the face equation (1) is:
   Z/20
and equation (2) is:
   Z*C*/[200*(100-PBF)]
(f) in the case of photoageing of the hands equation (1) is:
   Z/4
and equation (2) is:
   Z*C*/[40*(100-PBF)]
(g) in the case of skin cancer equation (1) is:
   Z/2.4
and equation (2) is:
   Z*C*/[20*(100-PBF)]
wherein:

Z is expressed in units of ng/ml and is the target local free (i.e. unbound) concentration that is expected to have a therapeutic effect against the skin condition. In the case of PPAR gamma agonists, Z should correspond to the peak plasma free drug concentration generated by dosing these drugs orally to treat effectively Type II diabetes mellitus. The appropriate value of Z may be determined experimentally in vivo by conventional known means using standard protocols.

C is expressed in units of ml/hr and is the rate of clearance of the drug substance. Clearance may be determined experimentally in vivo by conventional known means using standard tests. For example it can be determined from the plasma concentration in response to a defined input of drug using the equation given below:
Clearance (ml/hr)=Input rate (ng/hr)/Total plasma concentration (ng/ml)

PBF is the plasma bound fraction. Plasma bound fraction may be determined experimentally in vitro by conventional known means using standard tests (preferably a method employing equilibrium dialysis).

By “nominal flux” is meant the flux that passes through healthy skin in a healthy individual (i.e. not diseased skin) on a body site such as the trunk, abdomen, legs, back, arms (but not the face), especially the back, averaged over a 24 hour period. The equations take account of the different barrier and clearance properties of skin in disease conditions and on the face (for example the skin of the face is approximately 5 times more permeable than the skin of other parts of the body). The nominal flux of a given composition may be determined by an in vivo measurement or may be estimated using an in vitro measurement using the tests given below.

Since the nominal flux is a flux averaged over a 24 hr period it follows that the flux may be achieved through use of any suitable dose regimen (for example application a number of times per day, eg twice per day, or application every other day). It is expected that the same formulation will be applied on each treatment, although this is not required and, for example, one formulation could be applied at one time in the day and another formulation at another time in the day.

In the event that the calculated figure for Equation (2) is less than that for Equation (1) then the drug substance in question is not suitable for use in the method.

We have developed the above mentioned equations taking into account: the body surface area treated classically for the particular skin disease (for psoriasis and atopic dermatitis=10% body surface area (“BSA”), for facial acne, rosacea, photoageing of the hands and face=2% BSA, for skin cancer=1% BSA), the location in the skin of the target site(s) for the drug's effect for the particular skin disease, the nature of the barrier to drug absorption in the diseased state (for example whether is damaged or intact), relative absorption in different parts of the body (in particular that the face is notable for much higher absorption than the hands, back or elsewhere), and the extent of vascularisation by capillaries in the diseased state which can effect the rate of removal of drug into the system. The equations therefore reduce the needed flux only to consideration of three variables, defined above and labelled Z (a measure of intrinsic therapeutic activity), C (clearance) and PBF (a measure of availability of free drug).

Preferably the lower limit nominal flux is 10/3 times greater than the minimum nominal flux required to cause a therapeutic effect against the skin condition. Preferably the upper limit nominal flux is one-fifth of the maximum nominal flux which will cause a systemic anti-hyperglycaemic effect.

Preferably the lower limit nominal flux is 10/3 times than that given above in equations (1).

Preferably the upper limit nominal flux is one fifth of that given above in equations (2).

In a first embodiment of the invention the drug is farglitazar. It will be understood that farglitazar may be employed as a pharmaceutical acceptable salt eg the sodium salt, however weights given herein are calculated based on farglitazar free acid.

The value Z for farglitazar is 0.31 ng/ml [Peak plasma concentration for a 5 mg dose is about 157 ng/ml; 0.31 is 157*(100-99.8)/100].

The clearance of farglitazar is 15000 ml/hr (in adults)

The plasma bound fraction of farglitazar is 99.8%

The oral therapeutic dose for farglitazar is 2.5 mg in a single dose or two divided doses per day.

Thus there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment a pharmaceutical composition containing a farglitazar or a pharmaceutically acceptable salt thereof characterised in that the formulation delivers a nominal flux of farglitazar to the skin which is greater than A and less than B where A and B are as follows:

(a) in the case of psoriasis A is 0.13 (preferably 0.52) and B is 116;

(b) in the case of atopic dermatitis A is 0.052 and B is 39;

(c) in the case of facial acne A is 0.031 (preferably 0.16) and B is 116;

(d) in the case of rosacea A is 0.031 and B is 116;

(e) in the case of photoageing of the face A is 0.016 (preferably 0.078) and B is 116;

(f) in the case of photoageing of the hands A is 0.078 (preferably 0.39) and B is 581;

(g) in the case of skin cancer A is 0.13 (preferably 0.52, more preferably 2.6) and B is 1162;

(all figures are in units of ng/cm²/hr).

More preferably there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment a pharmaceutical composition containing a farglitazar or a pharmaceutically acceptable salt thereof characterised in that the formulation delivers a nominal flux of farglitazar to the skin which is greater than A and less than B where A and B are as follows:

(a) in the case of psoriasis A is 0.43 (preferably 1.7) and B is 23;

(b) in the case of atopic dermatitis A is 0.17 and B is 8;

(c) in the case of facial acne A is 0.1 (preferably 0.51) and B is 23;

(d) in the case of rosacea A is 0.1 and B is 23;

(e) in the case of photoageing of the face A is 0.051 (preferably 0.26) and B is 23;

(f) in the case of photoageing of the hands A is 0.26 (preferably 1.3) and B is 116;

(g) in the case of skin cancer A is 0.43 (preferably 1.7, more preferably 8.5) and B is 232;

(all figures are in units of ng/cm²/hr). In accordance with the invention we also provide the corresponding uses of formulations which deliver a nominal flux of farglitazar as recited above.

A further embodiment of the invention consists in varying the dose regimen of farglitazar or a pharmaceutically acceptable salt thereof delivered to the skin. PPAR gamma agonists deliver their full pharmacological potential over a relatively long treatment period that normally ranges from 4 to 18 weeks (J. J. Nolan et al, ‘Rosiglitazone taken once daily provides effective glycaemic control in patients with Type II diabetes mellitus’, Diabetic Medicine (2000) 17, 287-294; P. Raskin et al, ‘Rosiglitazone short-term monotherapy lowers fasting and post-prandial glucose in patients with Type II diabetes’, Diabetologia (2000) 43, 278-284; L. S. Phillips et al, ‘Once- and twice-daily dosing with rosiglitazone improves glycemic control in patients with Type II diabetes’, Diabetes Care (2001) 24(2), 308-315; P. Raskin et al., ‘A randomized trial of rosiglitazone therapy in patients with inadequately controlled insulin-treated Type II diabetes’, Diabetes Care (2001) 24(7), 1226-1232). The pharmacological benefit delivered is dose dependant. The rate at which this pharmacological benefit is delivered is also dose dependant. For example the clinical data generated by Raskin et al (2000) [P. Raskin et al, ‘Rosiglitazone short-term monotherapy lowers fasting and post-prandial glucose in patients with type II diabetes’, Diabetologia (2000) 43, 278-284] show that it takes about 8 weeks for a 2 mg b.i.d. rosiglitazone dosage regimen to achieve a reduction of fasting plasma glucose by 2 mmol/l, whilst it takes about 4 weeks for a 4 mg b.i.d. rosiglitazone dosage regimen to achieve the same results. Similar observations on the dose regimen compared with time to achieve equivalent benefit can be made in other studies (J. J. Nolan et al, ‘Rosiglitazone taken once daily porvides effective glycaemic control in patients with Type II diabetes mellitus’, Diabetic Medicine (2000) 17, 287-294; L. S. Phillips et al, ‘Once- and twice-daily dosing with rosiglitazone improves glycemic control in patients with Type II diabetes’, Diabetes Care (2001) 24(2), 308-315; P. Raskin et al., ‘A randomized trial of rosiglitazone therapy in patients with inadequately controlled insulin-treated type 2 diabetes’, Diabetes Care, (2001) 24(7), 1226-1232).

With oral administration, increasing the dose to reduce this time to get a defined pharmacological response can lead to unwanted side effects, for example an unwanted systemic pharmacological effect such as an anti-hyperglycaemic effect. However with topical administration, if the increased dose is locally tolerated, there is the opportunity to increase the dose without risking unwanted systemic side effects. It is therefore possible to deliver more drug than it would be normally required by a factor that will be dependant on the skin disease treated and dependant on the drug delivered. Based on the clinical results described above it is expected that for a PPAR gamma agonist, delivering a dose B 10 fold higher than a dose A, should decrease by 10 fold the time to get the same pharmacological response obtained after delivering dose A. This finding opens up the option of modulating dose regimen to reduce the time to get the same pharmacological response. To optimise the skin disease therapies considered in the present invention, from a patient safety or/and patient convenience point of view, PPAR gamma agonists can be administered in the following ways. The treatment can be divided into two parts. The first part of the treatment consists of delivering a high topical dose for a short time ranging from one day to 12 weeks, preferably ranging from one day to 2 weeks. The second part of the treatment consists of delivering a lower dose, either topically or orally for the remaining part of the treatment. If for the second part of the treatment, the drug is delivered orally, it is preferred not to exceed the established orally safe dosage regimen.

Thus according to a further aspect of the invention there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment a first pharmaceutical composition for a first period of time and a second pharmaceutical composition for a second period of time, each pharmaceutical formulation containing a drug substance which is farglitazar or a pharmaceutically acceptable salt thereof characterised in that the first formulation delivers to the skin a nominal flux of said drug substance which is greater than the minimum nominal flux required to cause a therapeutic effect against the skin condition (hereinafter “lower limit nominal flux”) and less than the minimum nominal flux which will cause a systemic anti-hyperglycaemic effect (hereinafter “upper limit nominal flux”) and the second formulation delivers a nominal flux of said drug substance to the skin which is greater than the lower limit nominal flux but is less than the nominal flux delivered for the first period of time.

There is also provided use of pharmaceutical compositions containing a drug substance which is farglitazar or a pharmaceutically acceptable salt thereof in the topical treatment to the skin of a patient suffering from a skin condition selected from the list consisting of psoriasis, atopic dermatitis, facial acne, rosacea, photoageing of the face, photoageing of the hands, and skin cancer characterised in that a first composition used for a first period of time delivers to the skin a nominal flux of said drug substance which is greater than the minimum nominal flux required to cause a therapeutic effect against the skin condition (hereinafter “lower limit nominal flux”) and less than the minimum nominal flux which will cause a systemic anti-hyperglycaemic effect and a second composition used for a second period of time delivers a nominal flux of said drug substance to the skin which is greater than the lower limit nominal flux but is less than the nominal flux delivered for the first period of time.

There is also provided use of pharmaceutical compositions containing a drug substance which is farglitazar or a pharmaceutically acceptable salt thereof in the manufacture of a medicament for the topical treatment to the skin of a patient suffering from a skin condition selected from the list consisting of psoriasis, atopic dermatitis, facial acne, rosacea, photoageing of the face, photoageing of the hands, and skin cancer characterised in that a first composition used for a first period of time delivers to the skin a nominal flux of said drug substance which is greater than the minimum nominal flux required to cause a therapeutic effect against the skin condition (hereinafter “lower limit nominal flux”) and less than the minimum nominal flux which will cause a systemic anti-hyperglycaemic effect and a second composition used for a second period of time delivers a nominal flux of said drug substance to the skin which is greater than the lower limit nominal flux but is less than the nominal flux delivered for the first period of time.

The values of the “lower limit nominal flux” and the “upper limit nominal flux” may be determined using the equations given above.

In accordance with the invention we also provide the corresponding uses of formulations which deliver a nominal flux of farglitazar as recited above.

More particularly there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment a first pharmaceutical composition for a first period of time and a second pharmaceutical composition for a second period of time, each pharmaceutical formulation containing a drug substance which is farglitazar or a pharmaceutically acceptable salt thereof, characterised in that the first formulation delivers a nominal flux of said drug substance to the skin which is greater than A and less than B, wherein A and B are as defined above, and the second formulation delivers a nominal flux of said drug substance to the skin which is greater than A as defined above but is less than the nominal flux delivered for the first period of time.

The first composition has a higher flux than the second composition in order to accelerate onset of therapeutic activity. The second composition of lower flux is intended to be suitable for maintenance therapy. Preferably the first period of time is a period of time long enough to establish onset of therapeutic activity, more particularly is long enough to establish at least 75% of maintenance level therapeutic activity. Typically the first period of time will be between 1 day and 12 weeks, preferably 1 day to 2 weeks. Preferably the second period of time is the remainder of the treatment period.

Preferably the second formulation delivers a nominal flux of drug substance to the skin which is greater than A as defined above but is less than the half the nominal flux delivered for the first period of time, more preferably less than one quarter nominal flux delivered for the first period of time.

According to another aspect of the invention there is provided a method for treating a skin condition selected from the list consisting of psoriasis, atopic dermatitis, facial acne, rosacea, photoageing of the face, photoageing of the hands and skin cancer which comprises administering topically to the skin of a patient in need of treatment for a first period of time a pharmaceutical composition containing a drug substance which is farglitazar or a pharmaceutically acceptable salt thereof characterised in that the formulation delivers to the skin a nominal flux of said drug substance which is greater than the minimum nominal flux required to cause a therapeutic effect against the skin condition (hereinafter “lower limit nominal flux”) and less than the minimum nominal flux which will cause a systemic anti-hyperglycaemic effect (hereinafter “upper limit nominal flux”) and subsequently administering the drug substance in an oral dosage form for a second period of time.

There is also provided use of a pharmaceutical composition containing a drug substance which is farglitazar or a pharmaceutically acceptable salt thereof in the topical treatment to the skin of a patient suffering from a skin condition selected from the list consisting of psoriasis, atopic dermatitis, facial acne, rosacea, photoageing of the face, photoageing of the hands, and skin cancer characterised in that the composition delivers to the skin for a first period of time a nominal flux of said drug substance which is greater than the minimum nominal flux required to cause a therapeutic effect against the skin condition and less than the minimum nominal flux which will cause a systemic anti-hyperglycaemic effect and said treatment is followed by administering said drug substance in an oral dosage form.

There is also provided use of a pharmaceutical composition containing a drug substance which is farglitazar or a pharmaceutically acceptable salt thereof in the manufacture of a medicament for the topical treatment to the skin of a patient suffering from a skin condition selected from the list consisting of psoriasis, atopic dermatitis, facial acne, rosacea, photoageing of the face, photoageing of the hands, and skin cancer characterised in that the composition delivers to the skin for a first period of time a nominal flux of said drug substance which is greater than the minimum nominal flux required to cause a therapeutic effect against the skin condition and less than the minimum nominal flux which will cause a systemic anti-hyperglycaemic effect and said treatment is followed by administering said drug substance in an oral dosage form.

The topical composition has a relatively high flux in order to accelerate onset of therapeutic activity. The oral dosage form is intended to be suitable for maintenance therapy. Preferably the first period of time is a period of time long enough to establish onset of therapeutic activity, more particularly is long enough to establish at least 75% of maintenance level therapeutic activity. Typically the first period of time will be between 1 day and 12 weeks, preferably 1 day to 2 weeks. Preferably the second period of time is the remainder of the treatment period. In this particular aspect, the oral dosage composition may be effective in causing a systemic anti-hyperglycaemic effect in patients sensitive to systemic PPAR gamma agonist therapy (eg patients suffering from Type II diabetes mellitus or who would otherwise benefit from insulin sensitivity enhancement).

Preferably the oral dosage form of farglitazar delivers 1.25 to 5 mg per day.

According to another aspect of the invention there are also provided formulations adapted for topical administration to the skin capable of delivering fluxes according to the methods described above.

Suitable formulations, the fluxes of which may be determined empirically using methods described herein, include ointments and solidified water/oil emulsion variants thereof, pastes, water/oil creams or lotions, oil/water creams or lotions, gels and rigid foams.

Ointments are hydrocarbon-based semisolid formulations containing dissolved or suspended drugs. They can, for example be prepared by incorporating high melting waxes into mineral oil (liquid petrolatum). Due to problems of drug solubility, and inconvenience associated with their greasy nature, it is often more convenient to incorporate an aqueous medium into the hydrophobic base to yield an emulsion. Examples of such aqueous mediums are the use of propylene glycol or Polyoxyethylene polymers (polyetheylene glycol). Pastes are ointments into which a high percentage of insoluble particulate solids have been incorporated.

Creams and lotions are semi-solid emulsion systems and the term is applied both to water/oil or oil/water.

The oil phase of an emulsion may comprise from 5-95% preferably from 5 to 40% of the composition by weight.

Gel formulations are semi-solid systems in which a liquid phase is trapped in a polymeric matrix of a natural or synthetic gum.

Rigid foams are systems in which air or some other gas is emulsified in a liquid phase to the point of stiffening.

If the composition contains an oily phase, excipients of this phase may comprise an oil based on animal, vegetable, mineral, silicone, fluorinated and/or synthetic oil.

Exemplary are the hydrocarbon oils such as; isohexadecane, paraffin oil or liquid petroleum jelly, petrolatum, microcrystalline wax, beeswax; perhydrosqualene, arara oil, sweet almond, calophyllum, palm, castor, avocado, jojoba, olive or cereal germ oil; alcohols such as oleyl alcohol, linoleyl or linolenyl alcohol, isostearyl alcohol, lanolin alcohol or octyl dodecanol.

Also exemplary are the silicone oils such as dimethicones, dimethiconols, cyclomethicones, silicone waxes like alkyl dimethicones or stearoxytrimethylsilane, cross-linked silicone elastomers as well as other polydimethylsiloxanes derivatives, optionally phenylated, such as phenyltrimethicones.

Also exemplary are the mono-, di- and triglycerides and their derivatives like caprylic/capric triglycerides.

Also exemplary are the esters such as methyl myristate, ethyl myristate, isopropyl myristate, butyl myristate, isobutyl myristate, methyl palmitate, ethyl palmitate, isopropyl palmitate, octyl palmitate, 2-ethylhexyl palmitate, cetyl palmitate, C12-15 alkyl benzoate, ethyl oleate, decyl oleate, butyl stearate, isopropyl isostearate, dioctyl adipate, diisopropyl adipate, 2-ethylhexyl hexanoate, 2-ethylhexyl 2-ethylhexanoate, ethyl laurate, isohexyl laurate, hexyl laurate, octyidodecyl octanoate, 2-ethylhexyl octanoate, isodecyl neopentanoate, isostearyl neopentanoate, myristyl propionate, ethylhexyl cocoate, 2-ethylhexyl caprate/caprylate, cocoyl-caprylate/caprate, propylene glycol dicaprylate/dicaprate, cetearyl isononanoate, cetostearyl isononanoate, isononyl isononanoate.

The compositions according to the invention may comprise, in addition:

(1) An emulsification agent for dispersing the oil phase, for example an anionic surfactant like carboxylic acids, carboxylic acids esters, sulfuric acid esters, sulfonic acids, amino acid amides; a cationic surfactant like polyoxyethylene alkyl amines, tetraalkyl ammonium salts; an amphoteric surfactant like phospholipids, N-alkyl amino acids, alkylamido alkylamines; a nonionic surfactants like fatty alcohols, alkoxylated fatty alcohols, polyoxyethylene-phenol esters, alkoxylated fatty acids, acyl sorbitans, polyoxyethylene-derivatives, acyl glycerides, polyoxyethylene alkyl amides, polyoxyethylene/polyoxypropylene block copolymers, polyoxyethylene silicone derivatives, alkylmethyl siloxane copolyol, alkyl-substituted polyvinyl polymers. In particular the emulsifying systems well known to the art and comprising of glyceryl stearate, glyceryl distearate, glyceryl oleate, propylene glycol stearate, glycol sterate, glyceryl stearate/PEG 100 stearate, sorbitan sesquioleate, cetyl alcohol, stearyl alcohol, sodium lauryl sulfate and cetomacrogol.

(2) An agent affecting the suspension of the oil phase, for example a copolymer of C.sub.10-C.sub.30 alkyl acrylates and acrylic or methacrylic acid or ester thereof; or an acrylamide/methylpropane-sulfonic acid copolymer.

(3) An agent for modifying its viscosity, and to provide textures which are gelled to a greater or lesser degree, such as: cellulose derivatives (carboxymethylcellulose, hydroxyethylcellulose, hydroxypropylmethylcellulose); natural gums such as xanthan, guar and carob gums, scleroglucans, chitin or chitosan derivatives, carrageenans; polycarboxyvinyl derivatives of the Carbomer type.

The compositions according to the invention may also comprise conventional additives and adjuvants for dermatological applications, such as preservatives, especially paraben esters like methylparaben, ethylparaben, propylparaben, butylparaben, or quaternary ammonium compounds like benzalkonium chloride, or formaldehyde donors like imidazonidinyl urea, or alcohols like benzyl alcohol, phenoxyethanol or acids like benzoic acid, sorbic acid; acids or bases used as pH buffer excipients; antioxidants, especially phenolic antioxidants like hydroquinone, tocopherol and derivatives thereof, as well as flavonoids, or miscellaneous antioxidants like ascorbic acid, ascorbyl palmitate; perfumes; fillers such as kaolin or starch; pigments or colorants; UV-screening agents; moisturizers, especially glycerin, butylene glycol, hexylene glycol, urea, hyaluronic acid or derivatives thereof; anti-free radical agents such as vitamin E or derivatives thereof; penetration enhancers especially propylene glycol; ethanol; isopropanol; dimethylsulfoxide; N-methyl-2-pyrrolidone; fatty acids/alcohols such as oleic acid, oleyl alcohol; terpenes such as limonene, menthol, 1-8 cineole; alkyl esters such as ethyl acetate, butyl acetate; ion pairing agents such as salicylic acid.

Example formulation bases include:

For ointments:

White petrolatum 95% (w/v), white wax 5%; or

White petrolatum 86% (w/w), stearyl alcohol 3%, white wax 8%, cholesterol 3%

White petrolatum 25% (w/w), stearyl alcohol 25%, propylene glycol 12%, sodium lauryl sulphate 1%, methylparaben 0.025%, propylparaben 0.015%, purified water 37%

Polyethylene glycol 4000 (Carbowax 4000) 50% (w/w), polyethylene glycol 400 50%

Propylene glycol 5% (w/w), mineral oil 69.5%, microcrystalline wax 25%, sorbitan sesquioleate 0.5%

For water/oil cream emulsions:

Oil phase: spermaceti 12.5% (w/w), white wax 12.0%, almond oil 55.58%

Aqueous phase: sodium borate 0.5% (w/w), purified water 19%

For oil/water cream emulsions (vanishing cream):

Oil phase: stearic acid 13% (w/w), stearyl alcohol 1%, cetyl alcohol 1%

Aqueous phase: glycerine 10% (w/w), methylparaben 0.025%, propylparaben 0.015%, potassium hydroxide 0.9%, purified water qs to 100%

For oil/water cream emulsions (general prototype):
Oil phase: stearic alcohol 15% (w/w), beeswax 8%, sorbitan monooleate 1.25%
Aqueous phase: 70% sorbitol solution 7.5% (w/w), polysorbate 80 3.75%, methylparaben 0.025%, propylparaben 0.015%, purified water qs to 100%

For paste:
Zinc oxide 25% (w/w), starch 25%, calamine 5%, white petrolatum qs to 100%

For gel:
Methocel 90 HC 4000 0.8% (w/w), Carbopol 934 0.24%, propylene glycol 16.7%, methylparaben 0.015%, sodium hydroxide qs to pH 7, purified water qs to 100%

Further details concerning suitable formulations may be obtained by reference to standard textbooks eg Modern Pharmaceutics (2002) vol 121 Published Marcel Dekker, Ed Banker and Rhodes or Harry's cosmeticology (2000), 8^{th} Edition, Chemical Publishing Co, Ed Gordon R.

Where needed for certain embodiments of the invention, drug substances for oral administration may, for example, be in solid oral dosage forms or liquid oral dosage forms. Example solid oral dosage forms include tablet or capsule form and may as necessary contain conventional excipients such as binding agents, fillers, lubricants, glidants, disintegrants and wetting agents. Unit dose compositions are preferred.

Examples of binding agents include acacia, alginic acid, carboxymethylcellulose calcium, carboxymethylcellulose sodium, dextrates, dextrin, dextrose, ethylcellulose, gelatin, liquid glucose, guar gum, hydroxyethyl cellulose, hydroxypropyl cellulose, hydroxypropyl methylcellulose, magnesium aluminium silicate, maltodextrin, methyl cellulose, polymethacrylates, polyvinylpyrrolidone, pregelatinised starch, sodium alginate, sorbitol, starch, syrup, tragacanth.

Examples of fillers include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol.

Examples of lubricants include calcium stearate, glyceryl monostearate, glyceryl palmitostearate, magnesium stearate, microcrystalline cellulose, sodium benzoate, sodium chloride, sodium lauryl sulphate, stearic acid, sodium stearyl fumarate, talc, zinc stearate.

Examples of glidants include colloidal silicon dioxide, powdered cellulose, magnesium trisilicate, silicon dioxide, talc.

Examples of disintegrants include alginic acid, carboxymethylcellulose calcium, carboxymethylcellulose sodium, colloidal silicon dioxide, croscarmellose sodium, crospovidone, guar gum, magnesium aluminium silicate, microcrystalline cellulose, methyl cellulose, polyvinylpyrrolidone, polacrilin potassium, pregelatinised starch, sodium alginate, sodium lauryl sulphate, sodium starch glycollate.

An example of a pharmaceutically acceptable wetting agent is sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

The nominal flux delivered by a given topical composition may be determined in vivo or in vitro. The in vivo method which employs healthy human volunteers is as follows:

The test composition is applied to the back of an individual at around 2 mg of formulation per cm². The area of the site treated must be measured. The site treated must be left opened for 2 hours prior to the individual wearing a cotton T-shirt for the remainder of the study. Plasma concentration measurements are made every 4 hours over a 24 hours period. The flux is calculated from the plasma level achieved using classical pharmacokinetics as described by Hadgraft et al (‘Investigations on the percutaneous absorption of the antidepressant rolipram in vitro and in vivo’-Hadgraft et al (1990) Pharm. Res. 7, 1307-1312).

Thus the Flux can be calculated using Equation A as follows: Equation A:

Flux (ng/cm²/hr) (mean over 24 hours)=Mean total plasma concentration over 24 hours (ng/ml)*total systemic clearance (ml/hr)/surface area (cm²) The alternative in vitro method involves an in vitro percutaneous study. The method which essentially follows Colipa recommendations (European Commission—99/III/COS/87—Basic Criteria for the in vitro assessment of percutaneous absorption of cosmetic ingredients—SCCNFP/0167/99/Final—23rd Jun. 1999) requires the use of opened diffusion cells (flow-through type, eg Permegear brand) that are temperature controlled to 32° C. (to mimic skin temperature) and which allow application of a thin film with an upper donor chamber and a lower receptor chamber. The diffusion cells should be sufficiently large to allow application of a clinically relevant dose, preferably greater than 2 cm² in area. A clinically relevant dose is considered to be around 2 mg of the formulation per cm² for the skin condition treated. If higher doses were to be applied, experiments proving linearity towards a clinical dose would be required. The human skin used should be dermatomed to approximately 300-500 μm and sourced from the back of the human. Skin batch integrity should be assessed with the use of a controlled solution of known flux (e.g. caffeine, methyl paraben . . . ). Drug follows through to a receptor mimicking well physiological condition and which does not contain solvents or other materials that would influence the integrity of the skin barrier. The preferred receptor is aqueous Phosphate Buffer Salined receptor. Study time is equal to about 24 hours, with periodic sample measurement and receptor replacement eg at 4 hour intervals.

Preferably the flux is determined by the in vivo measurement, although that it would be expected that the in vitro system is a satisfactory model for the in vivo system.

In a method according to the invention the skin condition of particular interest is psoriasis.

Other conditions of particular interest are atopic dermatitis, rosacea, facial acne and photoageing (hands and face), particularly facial acne and photoageing (hands and face).

As an aspect of the invention we also provide pharmaceutical compositions for use in the aforementioned methods and uses which comprise farglitazar or a pharmaceutically acceptable salt thereof and a physiologically acceptable carrier. In compositions for topical administration to the skin, the carrier will be physiologically acceptable on topical administration. For example, we provide a pharmaceutical composition comprising a drug substance which is farglitazar or a pharmaceutically acceptable salt thereof and a physiologically acceptable carrier characterised in that the formulation delivers to the skin a nominal flux of said drug substance which is greater than a lower limit nominal flux defined as the minimum nominal flux required to cause a therapeutic effect against the skin condition and less than an upper limit nominal flux defined as the minimum nominal flux which will cause a systemic anti-hyperglycaemic effect.

In the topical formulations according to the invention, the PPAR gamma agonist may be employed in combination with another therapeutic substance of benefit in topical administration, for example a corticosteroid (eg fluticasone propionate or mometasone furoate), a vitamin D3 derivative (eg calcipotriol), a retinoid (eg retinol, tazarotene, or trans- or cis-retinoic acid) or an immunomodulator (eg pimecrolimus or tacrolimus).

When used herein the expression “treatment” embraces treatment of established conditions as well as prophylaxis if appropriate
Farglitazar is preferably employed as the free acid or the sodium salt.

For the avoidance of doubt, all weights and amounts of farglitazar are based on the weight or amount of free acid, and when farglitazar is employed as a salt a correspondingly larger amount (to take account of the weight or amount of the salt) will be employed.

### EXAMPLES

All figures given below are percentages based on weight. Flux was measured using the in vitro method described above. The flux shown in the figures at time points 4, 8 hrs etc is the flux determined based on the concentration of drug substance in the receptor at the time point, and is thus the average flux between that time point and the previous one (i.e. the flux shown at 4 hrs is the average from 0-4 hrs)

### Example 1

Cream containing 0.1% of farglitazar₂₉

| | |
|---|---|
| Farglitazar | 0.1 |
| Buffer Salts (NaHPO₄/Na₂HPO₄/ | 1.0 |
| CH₃COOH) (pH = 4.3) | |
| Cetostearyl Alcohol | 7.3 |
| Sodium Lauryl Sulfate | 0.8 |
| Phenxoyethanol | 0.9 |
| White Soft Paraffin | 14.1 |
| Liquid Paraffin | 5.7 |
| Water | 70.1 |
| Total | 100.0 |

### Example 2

Cream containing 0.02% of farglitazar

| | Cream A |
|---|---|
| Farglitazar | 0.02 |
| Buffer Salts (NaHPO₄/Na₂HPO₄/ | 1.0 |
| CH₃COOH) (pH = 6.5) | |
| Cetostearyl Alcohol | 7.3 |
| Sodium Lauryl Sulfate | 0.8 |
| Phenxoyethanol | 0.9 |
| White Soft Paraffin | 14.1 |
| Liquid Paraffin | 5.7 |
| Water | 70.18 |
| Total | 100.0 |

### Example 3

Ointment containing 0.1% of farglitazar:

| | |
|---|---|
| Farglitazar | 0.1 |
| Propylene Glycol | 5.0 |
| Mineral Oil | 69.4 |
| Microcrystalline Wax 140-145 F. | 25.0 |
| Sorbitan sesquioleate | 0.5 |
| Total | 100.0 |

### BRIEF DESCRIPTION OF THE FIGURES

FIG. 1: Structure of mammalian skin

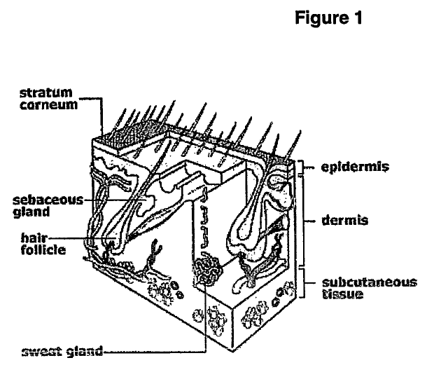

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

Above mentioned patents and patent applications are hereinbefore incorporated by reference.

1. A pharmaceutical composition suitable for topical administration to the skin which comprises farglitazar, or a pharmaceutically acceptable salt thereof, and a physiologically acceptable carrier.

2-3. (canceled)

4. A method of treatment of a skin condition selected from the group consisting of psoriasis, atopic dermatitis, facial acne, rosacea, photoageing of the face, photoageing of the hands and skin cancer, wherein said method comprises administering the composition according to claim 1.

5. A method for treating a skin condition selected from the group consisting of psoriasis, atopic dermatitis, facial acne, rosacea, photoageing of the face, photoageing of the hands and skin cancer, wherein said method comprises administering topically to the skin of a patient in need of treatment a pharmaceutical composition containing a drug substance which is farglitazar or a pharmaceutically acceptable salt thereof, wherein the pharmaceutical composition delivers to the skin a nominal flux of said drug substance which is greater than a lower limit nominal flux defined as the minimum nominal flux required to cause a therapeutic effect against the skin condition and less than an upper limit nominal flux defined as the minimum nominal flux which will cause a systemic anti-hyperglycaemic effect.

6-7. (canceled)

8. A method according to claim 5 wherein the lower limit nominal flux and the upper limit nominal flux are respectively given by equations (1) and (2) as follows:
(a) in the case of psoriasis equation (1) is:
Z/2.4
and equation (2) is:
Z*C/[200*(100-PBF)]
(b) in the case of atopic dermatitis equation (1) is:
Z/6
and equation (2) is:
Z*C*/[600*(100-PBF)]
(c) in the case of facial acne equation (1) is:
Z/10
and equation (2) is:
Z*C/[200*(100-PBF)]
(d) in the case of rosacea equation (1) is:
Z/10
and equation (2) is:
Z*C*/[200*(100-PBF)]
(e) in the case of photoageing of the face equation (1) is:
Z/20
and equation (2) is:
Z*C*/[200*(100-PBF)]
(f) in the case of photoageing of the hands equation (1) is:
Z/4
and equation (2) is:
Z*C*/[40*(100-PBF)]
(g) in the case of skin cancer equation (1) is:
Z/2.4
and equation (2) is:
Z*C*/[20*(100-PBF)]
wherein:
Z is expressed in units of ng/ml and is the target local free concentration of the drug substance that is expected to have a therapeutic effect against the skin condition;
C is expressed in units of ml/hr and is the rate of clearance of the drug substance; and
PBF is the plasma bound fraction of the drug substance.

9. A method according to claim 5 wherein the lower limit nominal flux and the upper limit nominal flux are respectively given by values A and B, expressed in units of ng/cm²/hr based on weight of drug as base, as follows:
(a) in the case of psoriasis A is 0.13 and B is 116;
(b) in the case of atopic dermatitis A is 0.052 and B is 39;
(c) in the case of facial acne A is 0.031 and B is 116;
(d) in the case of rosacea A is 0.031 and B is 116;
(e) in the case of photoageing of the face A is 0.016 and B is 116;
(f) in the case of photoageing of the hands A is 0.078 and B is 581;
(g) in the case of skin cancer A is 0.13 and B is 1162.

10. A method for treating a skin condition selected from the group consisting of psoriasis, atopic dermatitis, facial acne, rosacea, photoageing of the face, photoageing of the hands and skin cancer, wherein said method comprises administering topically to the skin of a patient in need of treatment a first pharmaceutical composition for a first period of time and a second pharmaceutical composition for a second period of time, each pharmaceutical formulation containing a drug substance which is farglitazar or a pharmaceutically acceptable salt thereof, wherein the first pharmaceutical composition delivers to the skin a nominal flux of said drug substance which is greater than a lower limit nominal flux defined as the minimum nominal flux required to cause a therapeutic effect against the skin condition and less than an upper limit nominal flux defined as the minimum nominal flux which will cause a systemic anti-hyperglycaemic effect and the second pharmaceutical composition delivers a nominal flux of said drug substance to the skin which is greater than the lower limit nominal flux but is less than the nominal flux delivered for the first period of time.

11-12. (canceled)

13. A method for treating a skin condition selected from the group consisting of psoriasis, atopic dermatitis, facial acne, rosacea, photoageing of the face, photoageing of the hands and skin cancer, wherein said method comprises administering topically to the skin of a patient in need of treatment for a first period of time a pharmaceutical composition containing a drug substance which is farglitazar or a pharmaceutically acceptable salt thereof, wherein the pharmaceutical composition delivers to the skin a nominal flux of said drug substance which is greater than a lower limit nominal flux defined as the minimum nominal flux required to cause a therapeutic effect against the skin condition and less than an upper limit nominal flux defined as the minimum nominal flux which will cause a systemic anti-hyperglycaemic effect and subsequently administering the drug substance in an oral dosage form for a second period of time.

14-15. (canceled)

16. A method according to claim 10, wherein the farglitazar in the pharmaceutical composition is present as 2(S)-(2-benzoyl-phenylamino)-3-{4-[2-5-methyl-2-phenyl-oxazol-4-yl)-ethoxy]-phenyl}-propionic acid, or as a sodium salt thereof.

17. A method according to claim 5, wherein the skin condition is psoriasis.

18. A method according to claim 5, wherein the skin condition is facial acne.

19. A method according to claim 5, wherein the skin condition is photoageing of the face or photoageing of the hands.

20-21. (canceled)

22. A method according to claim 13, wherein the farglitazar in the pharmaceutical composition is present as 2(S)-(2-benzoyl-phenylamino)-3-{4-[2-5-methyl-2-phenyl-oxazol-4-yl)-ethoxy]-phenyl}-propionic acid, or as a sodium salt thereof.

23. A method according to claim 10, wherein the skin condition is psoriasis.

24. A method according to claim 10, wherein the skin condition is facial acne.

25. A method according to claim 10, wherein the skin condition is photoageing of the face or photoageing of the hands.

26. A method according to claim 13, wherein the skin condition is psoriasis.

27. A method according to claim 13, wherein the skin condition is facial acne.

28. A method according to claim 13, wherein the skin condition is photoageing of the face or photoageing of the hands.

29. 2(S)-(2-Benzoyl-phenylamino)-3-{4-[2-5-methyl-2-phenyl-oxazol-4-yl)-ethoxy]-phenyl}-propionic acid sodium salt.

30. A pharmaceutical composition comprising 2(S)-(2-benzoyl-phenylamino)-3-{4-[2-5-methyl-2-phenyl-oxazol-4-yl)-ethoxy]-phenyl}-propionic acid sodium salt and a physiologically acceptable carrier.
